(19) United States
(12) Patent Application Publication
(10) Pub. No.: US 2004/0171988 A1
(43) Pub. Date: Sep. 2, 2004

(54) Combine needle and dilator device for central venous and arterial catheterization

(76) Inventor: Pierino Moretti, Bastia Di Rovolon (IT)

Correspondence Address: HARRISON & EGBERT, 412 MAIN STREET, 7TH FLOOR, HOUSTON, TX, 77002, US

(21) Appl. No.: 10/476,840

(22) PCT Filed: Feb. 26, 2003
(86) PCT No.: PCT/IT03/00108

(30) Foreign Application Priority Data
Mar. 8, 2002 (IT) PD2002 A 000060
Dec. 17, 2002 (IT) PD2002 A 000323

(51) Int. Cl. A61M 5/178

(52) U.S. Cl. 604/164.01

(57) ABSTRACT

This is a disposable device for arterial or central venous catheterization, comprising a needle (A) connected to a piston (P) perforated in the axial direction to allow for the passage of a Seldinger wire; inserted in a dilator (D) with a tubular container (I) at the end. The container (I) is complete with a lateral aspirator (S), that communicates with the inside of the piston (P) and with the hole in the needle (A) to allow for the position of the needle in the vein to be verified, and where said communication is occluded when said piston (P) is drawn back or partially withdrawn from the container (I). The device is also complete with a Seldinger wire (F) with a corresponding advancement mechanism (Fm).

### RELATED U.S. APPLICATIONS

Not applicable.

### STATEMENT REGARDING FEDERALLY SPONSORED RESEARCH OR DEVELOPMENT

Not applicable.

### REFERENCE TO MICROFICHE APPENDIX

Not applicable.

### FIELD OF THE INVENTION

This patent has to do with percutaneous central venous and arterial catheterization for anesthetization or the administration of medication; in particular, it concerns a combined needle and dilator device for the insertion of a percutaneous catheter in a major central vein or artery.

### BACKGROUND OF THE INVENTION

Percutaneous catheterization of major central veins and arteries is currently a very common practice, representing an important, essential part of the proper performance of numerous therapeutic procedures in both hospitalized cases and outpatients, e.g. for pressure monitoring, anesthesia and the administration of medication in difficult circumstances.

In anesthetization or the administration of drugs, the veins become stiff and it is consequently necessary to take action through a major central vein, such as the jugular or the subclavian.

Percutaneous catheterization of a major central vein currently involves the use of various surgical and medical instruments, as well as a procedure based on a precise protocol, the main stages of which are summarized below.

After disinfecting and locally anesthetizing the operating field, the skin is punctured with a syringe to reach the blood vessel, aspirating a minimal quantity of blood to make sure that the vein or artery has been accessed.

A Seldinger guide wire is inserted through an access hole provided in the syringe's piston.

A suitable length of guide wire is advanced into the vein or artery until it reaches the catheter position required.

Leaving the Seldinger wire in place, the syringe is then withdrawn and replaced with a dilator, inserted on the same guide wire, comprising a rigid cannula designed to expand the path from the skin to the blood vessel.

The dilator is subsequently withdrawn and the catheter is introduced in its place, making it slide over the Seldinger wire until it penetrates the blood vessel and reaches the required position at the end of the wire.

Finally, the Seldinger wire is withdrawn through the inside of the catheter, which can then be connected to the bottles of pharmaceutical and/or anesthetic solution, as the case may be.

This procedure calls for the use of several surgical and medical instruments and has a number of drawbacks and problems.

Different instruments are used, each of which affects the catheter installation site and all of which must be handled with great care and attention.

Each instrument is handled by several operators (assistant, nurse, physician) and has to be introduced and then withdrawn, consequently producing microtraumas along the course of the catheter, with the risk of each instrument tilting to one side.

The removal of each instrument to allow for the insertion of the next exposes the catheter installation site to the environment, with a consequent risk of infection.

If the catheter has to be inserted in the jugular vein, this means placing it in the neck region where the operator's range of action is restricted, making all the above-described steps very complicated.

Many of the above-mentioned procedures demand measured insertions and withdrawals, since there is a risk, for instance, of puncturing the blood vessel wall or, worse still, of inserting the Seldinger wire and/or catheter too deeply, consequently damaging organs or membranes, such as the lung or peritoneum.

To avoid all the above-mentioned drawbacks and problems, a new combined needle and dilator device has been designed and developed for use in central venous or arterial catheterization.

One of the main aims of the new device is to contain the risks deriving from the handling of the apparatus by more than one person.

Another aim is to be able to ascertain the correct positioning of the needle by immediately ascertaining the (pale or dark) color of the blood aspirated after inserting the needle in the vessel.

Another aim of the new device is to substantially reduce the skin and muscle lesions deriving from the insertion and withdrawal of the various instruments.

A further aim of the new device is to limit the continuous exposure of the catheter access site.

Another aim of the new device is to facilitate the insertion of the catheter even in difficult situations, such as in the neck.

A further aim of the new device is to limit any voluntary or involuntary tilting of the various instruments during the catheterization procedure.

Another aim of the new device is to reduce the number of stages and the time it takes to insert the catheter, i.e. to eliminate dead times.

Another aim of the new device is to improve the efficiency of each instrument and consequently of the catheterization procedure as a whole.

### BRIEF SUMMARY OF THE INVENTION

These and other direct and complementary aims are achieved by the new combined needle and dilator device for venous or arterial catheterization comprising a piston with a needle at one end that slides inside a container, the latter being equipped at one end with a dilator and a lateral aspirator device, and a Seldinger guide wire with a mechanism for its advancement, combined and integrated with each other.

The needle is attached to the end of the piston, the other end of which supports the mechanism for advancing the Seldinger wire, that slides inside the needle.

The needle and piston are housed inside a container equipped at one end with a dilator, so that the piston slides inside the container and the needle slides inside the dilator.

The needle is attached to the end of the piston, the other end of which can support the mechanism for advancing the Seldinger wire, which slides inside the needle.

The needle and piston are housed inside a container fitted at the end with a cannula acting as a dilator, so that the piston slides inside the container and the needle slides inside the dilator cannula.

The syringe-like aspirator is attached to the side of the container preferably in the vicinity of the connection between the needle and the piston.

The piston contains a chamber or space, preferably in line with the rear end of the needle, that places the inside of the needle in communication with the aspirator situated alongside when the piston is in its advanced position.

The characteristics of the new combined needle and dilator device for central venous or arterial catheterization will be better explained in the following description, with reference to the drawings attached, provided only as an illustrative and not restrictive example.

### BRIEF DESCRIPTION OF THE SEVERAL VIEWS OF THE DRAWINGS

FIGS. 1, 2 and 3 respectively show two cross sections and one elevation of the preferred embodiment of the new device.

FIG. 1a shows the version for central venous catheterization, while FIG. 1b shows the version for arterial catheterization.

FIGS. 4 and 5 respectively show a cross-section of the combined and separate parts of a second embodiment of the new device.

### DETAILED DESCRIPTION OF THE INVENTION

The new combined needle and dilator device for central venous or arterial catheterization essentially comprises: a container (1) with a dilator (D), a needle (A) with a piston (P), an aspirator (S), a Seldinger wire (F) with a mechanism for its advancement (Fm).

The parts of the new device are joined to form a single object so as to enable their function and synergic use.

The needle (A) has a sharp front tip (Ai) and a rear end (Ap) attached to the piston (P).

Said needle (A) is long enough to enable it to reach as far as the blood vessel required.

The piston (P) is a cylindrical element complete with a chamber or space (Pa) that places the tip (Ap) of the needle (A) in communication with the end of the piston (P).

The piston (P) is fitted with a cursor (Pr) or other appendage to enable the operator to displace the piston (P).

A further hole (Pf) inside the piston (P) places the tip (Ai) of the needle (A) in communication with the outside, or it can be used for threading the Seldinger wire (F).

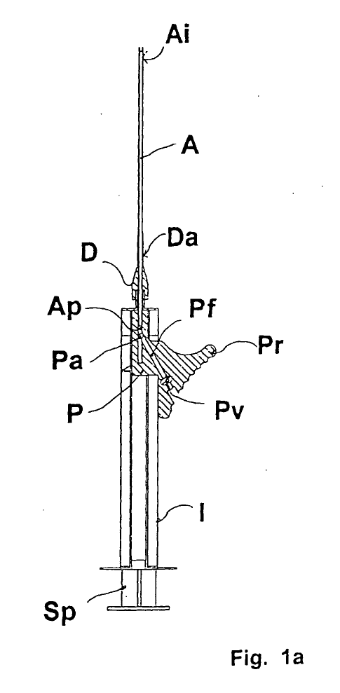

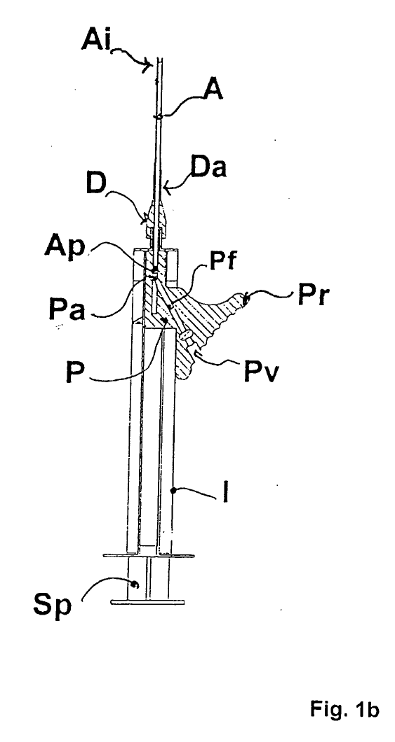

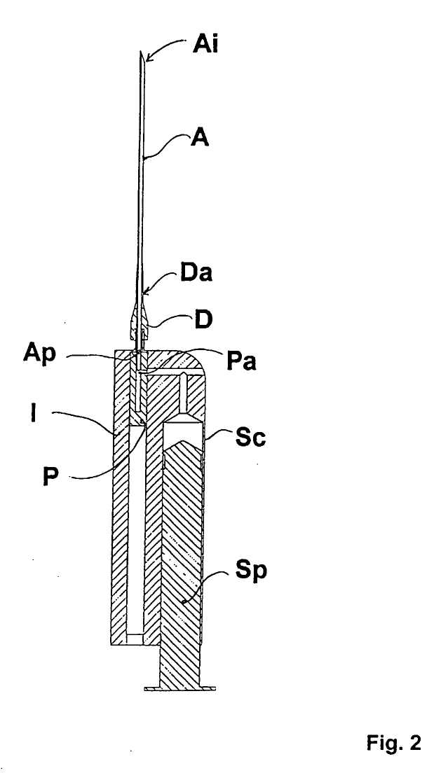

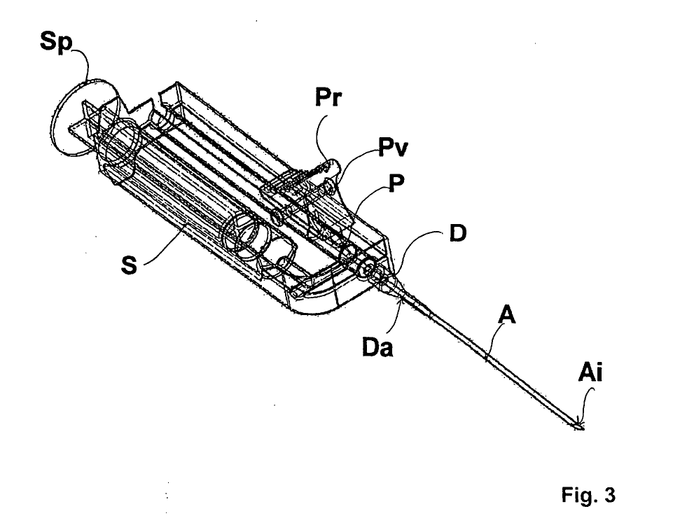

If the hole (Pf) in the piston (P) places the needle (A) in communication with the cursor (Pr) on the piston (P) (FIGS. 1, 2, 3), a sliding valve (Pv) is provided for opening or closing the communication between said hole (Pf) and the outside.

Said needle (A) and piston (P) are housed and slide respectively inside the dilator (D) and container (1).

The dilator (D) is composed of a cannula with an outside diameter comparable with the diameter of the catheter being inserted and has a hole inside of sufficient diameter to contain the needle (A).

In particular, the length of the dilator (D) is shorter than the length of the needle (A).

One end (Da) of the dilator (D) is conical so as to form a connection between the needle (A) and the cross section of the dilator (D).

The other end (Di) of the dilator (D) is attached to the container (1).

Said container (1) is a tubular body, generically coaxial to the dilator (D) and suitable for housing the piston (P), so that the needle (A) can be contained and slide within the dilator (D).

The container (1) has an internal diameter sufficient to allow for the sliding of the piston (P) and long enough to ensure that the cursor (Pr) on the piston (P) lies outside the container, or in any case that the piston can be displaced, making the needle (A) withdraw completely inside the dilator (D).

The aspirator (S) is attached to the side of the container (1).

Said aspirator (S) has the typical shape of a syringe, i.e. a cylinder (Sc) with a mandril (Sp) sliding inside.

Said aspirator (S) lies preferably parallel to the container (1) and communicates with the inside of the container (1) in the region in the vicinity of the dilator (D), i.e. in line with the chamber or space (Pa) in the piston (P) communicating with the hole (Ap) in the needle (A). To be more precise, the communicating space between the aspirator (S) and the inside of the container (1) is restricted to the area where the chamber or space (Pa) in the piston (P) coincides with said piston (P) being inserted completely in the container (1).

The chamber or space (Pa) in the piston (P) is occluded by the container (1) when said piston (P) is drawn back to make the needle (A) withdraw inside the dilator (D).

The negative or positive pressure exerted by the mandril (Sp) in the aspirator (S) thus only gives rise to a positive or negative pressure inside the needle (A) if the piston is completely inserted in the container (1), thanks to the communication between the hole in the needle (A) and the mandril (Sp) permitted by the chamber (Pa) in the piston (P).

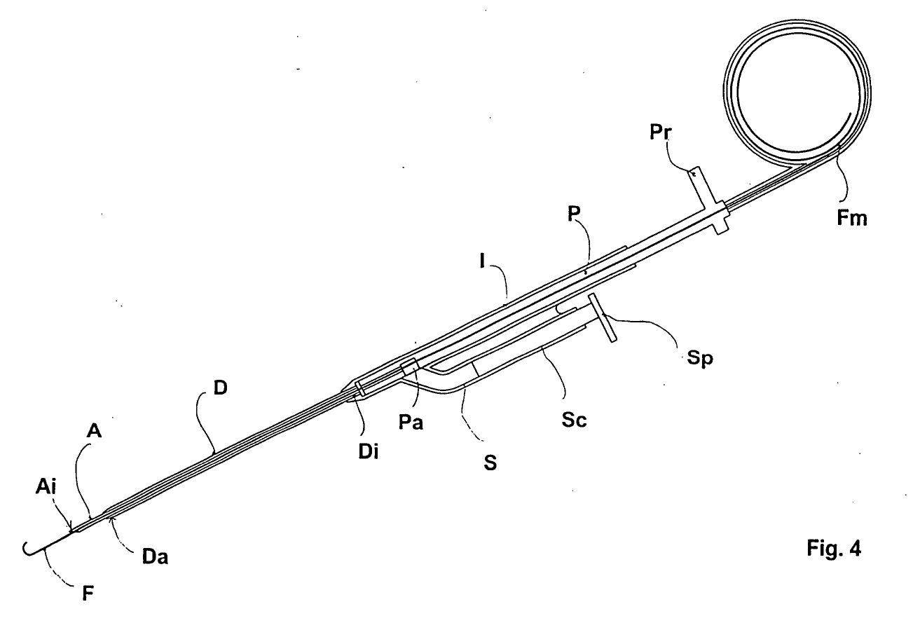

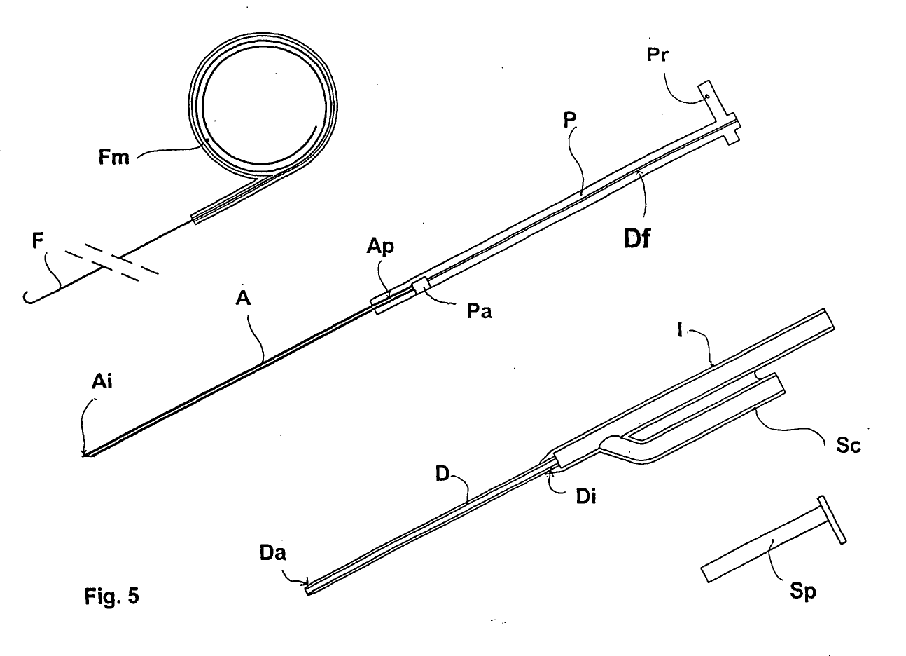

The second embodiment (FIGS. 4 and 5) also shows the Seldinger guide wire (F), which is a metal wire contained inside a plastic sheath so that it is flexible, radio-opaque and sterile.

Said Seldinger wire is surrounded by the mechanism for its advancement (Fm), which is in turn attached to the piston (P) so that the Seldinger wire (F) can be threaded through the hole (Pf) in the side or end of the piston (P) and run through all or part of the piston to pass into the needle (A).

The advancement mechanism (Fm) enables the Seldinger wire (F) to be advanced or withdrawn through the piston (P) and needle (A).

Said advancement mechanism (Fm) for advancing the Seldinger wire (F) is connected to the piston (P) in such a way that it can be separated from the piston (P).

Said advancement mechanism (Fm) is shaped in such a way that the degree of advancement of the Seldinger wire (F) can be accurately monitored and controlled, and to enable the guide wire (F) to be withdrawn from the mechanism (Fm) by hand.

The above-described parts are joined together to form a new combined needle and dilator device for arterial and central venous catheterization:

the container (1) with the dilator (D) is solidly attached to the aspirator (S) complete with a mandril (Sp);

the piston (P) and needle (A) are inserted in the container (1) so that the piston (P) fits into the container (1) and the needle (A) fits into the dilator (D); and

the guide wire (F) advancement mechanism (Fm) is attached to the end of the piston (P) opposite the needle (A), so that the Seldinger wire (F) lodges at least partially inside the piston (P) and inside the needle (A) (depending on the embodiment, FIGS. 4, 5).

The new combined needle and dilator device for arterial or central venous catheterization, as described above, ensures a more straightforward and accurate catheterization procedure.

The new device requires the performance of the main operating stages described below, but not to be considered as binding.

Initially, all parts of the new device are combined together, as described above, with the piston (P) completely inserted in the container (1) so that the needle (A) extends from the dilator (D), with the mandril (Sp) in the aspirator (S) completely pressed inside the aspirator (S), and with the Seldinger wire (F) inserted only inside the piston (P).

The new device is applied to the skin so that the needle (A) penetrates far enough to reach the vein. At the same time, a mild negative pressure is exerted on the mandril (Sp) of the aspirator (S) so as to aspirate blood from the vein concerned, which pours out through the needle (A) and through the chamber or space (Pa) in the piston (P) to reach the aspirator (S). Said aspiration procedure enables the operator to ensure that the blood vessel has been accessed and to ascertain its nature (vein or artery).

As soon as the needle (A) has reached the blood vessel concerned, the Seldinger wire (F) is advanced through the needle (A) and inside the blood vessel until its tip reaches the required position inside the vein concerned.

Then the container (1) and dilator (D) are moved forward and the piston (P) and needle (A) are withdrawn, so that the tip (Di) of the dilator (D) reaches the blood vessel and expands the path covered by the needle (A) to the diameter of the catheter being inserted.

The entire new device is then removed except for the Seldinger wire (F), which remains in situ.

The catheter to insert is threaded onto the Seldinger wire (F) until it reaches the tip of the guide wire (F) inside the blood vessel.

Finally, the Seldinger wire (F) is withdrawn through the catheter, which is now in the required position and ready for connection to the bottles of pharmaceutical and/or anesthetic solution, as the case may be.

The new combined needle and dilator device for arterial or central venous catheterization, as described above, offers numerous advantages.

All the instruments needed are combined into a single device that is grasped and handled by the physician, eliminating all the drawbacks typical of the serial insertion and removal of several separate instruments.

All the steps in the procedure can be carried out without any risk of tilting any of the instruments sideways.

All microtraumas at the catheter installation site deriving from the insertion and removal of each instrument are prevented because the needle (A) and the dilator (D) form a single combined needle-cum-dilator element and their relative reciprocal displacement makes the previous two distinct actions unnecessary.

The catheter insertion point is exposed to the environment only for a brief instant, after removing the new device and shortly before inserting the catheter, thus avoiding any contamination of the site.

If the catheter has to be inserted in the jugular vein in the neck region, all the steps are performed with a single device that is applied at the beginning of the procedure and removed at the end of the procedure.

Since all the instruments are combined into a single device, any incorrect movements (tilting, excessively deep insertions, involuntary withdrawals, etc.), that might interfere with the efficiency and safety of the catheter in situ, are avoided.

The new device is preferably of the disposable type to ensure perfect sterile conditions for every catheter insertion procedure.

Therefore, with reference to the above description and to the enclosed figures, the following claims are made.

I claim:

1. Combined device for blood vessel catheterization characterized in that it includes:
a needle (A) perforated axially and connected at one end (Ap) to a piston (P) complete with a hole (Pf) for the passage of a Seldinger wire;
a dilator (D) with a tubular container at one end (1);
wherein said needle (A) with piston (P) are inserted respectively in the dilator (D) and container (1) so that the needle (A) lodges and substantially slides inside the dilator (D) and the piston (P) lodges and slides inside the container (1).

2. Combined device as in claim 1, characterized in that said dilator (D) comprises a cannula shorter than the needle (A), with an outer diameter comparable with the diameter of the catheter being inserted, and it has a hole inside of diameter sufficient to contain the needle (A), and where the end (Da) of said dilator (D) is conical so as to form a connection between the needle (A) and the cross-section of the dilator (D).

3. Combined device as in claims 1 and 2, characterized in that the container (1) partially or entirely contains the piston (P), and where said piston (P) is complete with a cursor (Pr) or other appendage that enables the operator to move the piston (P) inside the container (1).

4. Combined device as in claims 1, 2, 3, characterized in that the piston (P) has a valve (Pv) that opens and closes the hole (Pf) for inserting the Seldinger wire.

5. Combined device as in claim 4, characterized in that said valve is of the lateral sliding type.

6. Combined device as in claims 1, 2, 3, 4, 5, characterized in that the piston (P) is complete with a chamber or space (Pa) that places said hole in the needle (A) in communication with the outside of the piston (P).

7. Combined device as in claims 1, 2, 3, 4, 5, 6, characterized in that the container (1) is fitted laterally with an aspirator (S), preferably lying alongside the container (1), that communicates with the inside of the container (1).

8. Combined device as in claims 1, 2, 3, 4, 5, 6, 7, characterized in that the aspirator (S) has the typical shape of a syringe, i.e. a cylinder (Sc) with a mandril (Sp) sliding inside, and where said aspirator (S) communicates with the inside of the container (1), with the chamber or space (Pa) in the piston (P), and with the hole in the needle (A).

9. Combined device as in claims 1, 2, 3, 4, 5, 6, 7, 8, characterized in that the aspirator (S) communicates with the space or chamber (Pa) in the piston (P) to ascertain the position of the needle in the vein, and where said communication is occluded when said piston (P) is drawn back or partially withdrawn from the container (1).

10. Combined device as in claims 1, 2, 3, 4, 5, 6, 7, 8, 9, characterized in that it is complete with a Seldinger wire (F) with a corresponding advancement mechanism (Fm), and where said advancement mechanism (Fm) is attached in a removable manner to the end of the piston (P), so that the Seldinger wire (F) is threaded through the hole (Pf) in the piston (P) and thus passes into the needle (A).

11. Combined device as in claim 10, characterized in that said advancement mechanism (Fm) is designed to enable the degree of advancement of the Seldinger wire (F) to be exactly verified and controlled.
